(19) United States
(12) Patent Application Publication
(10) Pub. No.: US 2007/0232918 A1
(43) Pub. Date: Oct. 4, 2007

(54) Doppler helmet

(76) Inventor: William Taylor, San Antonio, TX (US)

Correspondence Address: Daniel D. Chapman;Jackson Walker, LLP, Suite 2400, 112 E. Pecan, San Antonio, TX, 78205, US

(21) Appl. No.: 11/787,963

(22) Filed: Apr. 18, 2007

Related U.S. Application Data
(63) Continuation-in-part of application No. 11/005,514, filed on Dec. 6, 2004.

(51) Int. Cl. A61B 8/00

(52) U.S. Cl. 600/453

(57) ABSTRACT

A helmet manufactured for one specific person, made from rigid synthetic materials, to specifications determined by data obtained from a previously obtained MRI (magnetic resonance imaging) scan of that person's brain, intra-cranial arteries, and skull. The helmet and its attached adapters hold in place various Doppler probes directed at specific arteries, both intra-cranial and extra-cranial, to provide continuous readings of the velocity of the blood flow through those arteries.

This application is a continuation-in-part application to and claims priority to U.S. patent application Ser. No. 11/005,514, filed Dec. 6, 2004, which is incorporated by reference herein.

### FIELD OF THE INVENTION

Doppler helmets, more particularly, Doppler helmets custom fit to the head with the further addition of securing pins for fastening and pins to positively locate the helmet adjacent the skull of the user and a method for using the same.

### BACKGROUND OF THE INVENTION

When a patient suffers a subarachnoid hemorrhage (SAH), he is at risk of developing vasospasm in some of the major intracranial arteries at some time during the first two weeks after the bleed. When vasospasm occurs, the muscle wall of the artery contracts, narrowing the lumen, and restricting the blood flow. Prolonged vasospasm will cause a stroke in that part of the brain relying on this artery. The current management of vasospasm is somewhat risky, and is not initiated in the absence of true vasospasm, but should be initiated as soon as it is detected. Whereas current practice involves intermittent manual monitoring, the helmet (1) can be set up for automatic constant monitoring.

Monitoring of the intracranial blood flow as well as extracranial blow flow is essential. Those physiological factors that influence heart rate and blood flow generally affect both intracranial and a extra-cranial arteries similarly. Vasospasm from SAH does not affect the extra-cranial arteries. Because of this, the rise in the ratio of the flow rates, of intracranial to extra-cranial arteries, is the best indication of active vasospasm. Applicant's helmet (1) allows for monitoring both. The monitor can easily be programmed to constantly calculate the ratio and set off an alarm if the ratio exceeds a predetermined level.

It must be noted that current Doppler technology does not adequately penetrate an intact skull, except over the temporal bones, where the skull is thin. The device described here would necessitate making burr holes in the patient's skull along the flow vectors of the major intracranial arteries. The precise position for these burr holes can be determined at the time of the initial digital scan, and the scalp can be marked appropriate for later craniostomies.

The object of this invention is to provide a means to maintain a Doppler probe (2) in an exact position, with respect to a patient's intracranial arteries, to continuously monitor blood flow in those arteries.

### BRIEF SUMMARY OF THE INVENTION

This application provides for a helmet that is custom-made for one person. This helmet is typically made of a rigid plastic material to specifications dictated by the data obtained from a digital scan of the person's head, such as a CAT scan or MRI scan. The helmet is designed to be secured to the user's skull by, typically, four skull pins threaded through the helmet, and seated on the user's skull. The helmet is constructed with a plurality of windows, through which Doppler monitoring probes will be fitted. When properly affixed to the user's head, the windows will be positioned such that the flow vectors of the major intracranial arteries of the user will be directed through the windows and thus at the Doppler monitoring probes. In order to fix the helmet to the head in the correct position, the helmet typically will be made with orientation lines scribed on the surface that will point to a plurality of easily recognized landmarks on the user's head, i.e., internal auditory canal and medial canthus of the eye. The data needed for placement of the windows and the orientation lines is provided by the initial digital scan.

### BRIEF DESCRIPTION OF THE SEVERAL VIEWS OF THE DRAWINGS

FIG. 1 is an elevational view of the front of the helmet (1) showing it fitted to the user with a Doppler monitor probe (2) mounted to an adapter (3), attached to the helmet over the left temporal bone, and a representation of the left internal carotid artery (4) with left middle cerebral (5) and left anterior cerebral branches (6) (ghosted in).

FIG. 1A illustrates the view of a window (20) with the Doppler probe in place.

FIG. 2 is an elevational view of the right side of the helmet (1) showing it fitted to the user with a Doppler monitor probe (2) mounted to an adapter (3), attached over the right temporal bone, and a representation of the right internal carotid artery (7) with the right middle cerebral (8) and right anterior cerebral (9) branches (ghosted in).

FIG. 3 is an elevational view of the front of the helmet (1) fitted to the user with a Doppler monitor probe (2) mounted to an adapter (3) attached over the apex and a representation of the basilar artery (10) and some of its branches (11) (ghosted in).

FIG. 4 is an elevational view of the right side of the helmet (1) fitted to the user with a Doppler monitor probe (2) mounted to an adapter (3) attached over the apex and a representation of the basilar artery (10) and some of its branches (11).

FIG. 5 is an elevational view of the right side of the helmet (1) fitted to the user with a Doppler monitor probe (2) mounted to an adapter (12) attached to the lower edge of the helmet (1) and with a representation of the extra-cranial portion of the internal carotid artery.

FIG. 6 is a view of the right side of the helmet (1) fitted to the user showing the orientation lines (14) inscribed on the helmet directed at anatomical landmarks.

FIG. 7 is a typical Doppler probe (2).

FIG. 8 is a Doppler probe (2) secured to an adapter (3) that attaches to the helmet (1) (not shown).

FIG. 9 is a Doppler probe (2) secured to the adapter (12) that attaches (with fasteners, not shown) to the helmet (1) over the extra-cranial portion of the internal carotid artery (see FIG. 5).

FIG. 10 is a view of the right side of the helmet (1) fitted to the user showing two skull pins (15) threaded through the helmet (1).

FIG. 11 is a view of the top of the helmet (1) fitted to the user showing four skull pins (15) threaded through the helmet (1).

FIG. 12 is a cross-section view of the helmet (1) showing a skull pin (15) threaded through the helmet (1).

FIG. 13 is a cross-section view of the skull (16) with a skull pin (15) seated on the skull (16).

FIG. 14 is a view of an adapter (17) affixed to a window in the helmet (1) and upon which the Doppler probe (2) will be mounted.

FIG. 15 is a view of an adapter to adjustable locate a Doppler probe.

FIG. 16 illustrates burr holes drilled in the skull aligned along the Doppler probe axis.

### DETAILED DESCRIPTION OF THE INVENTION

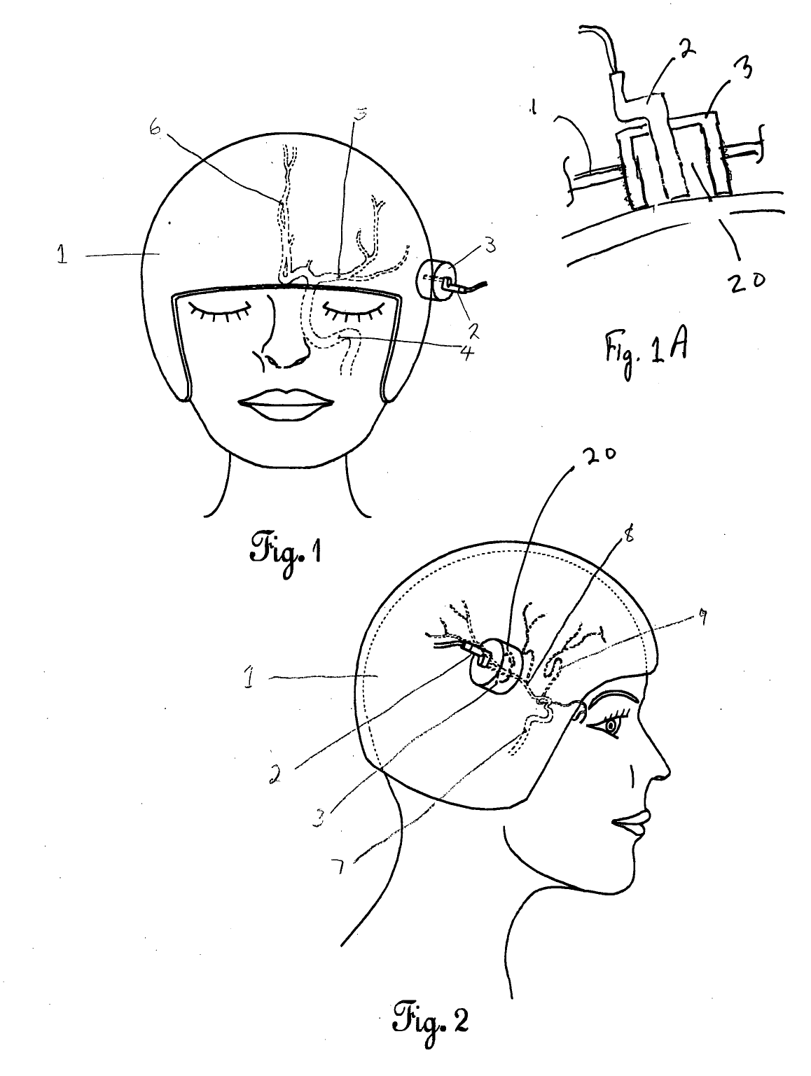

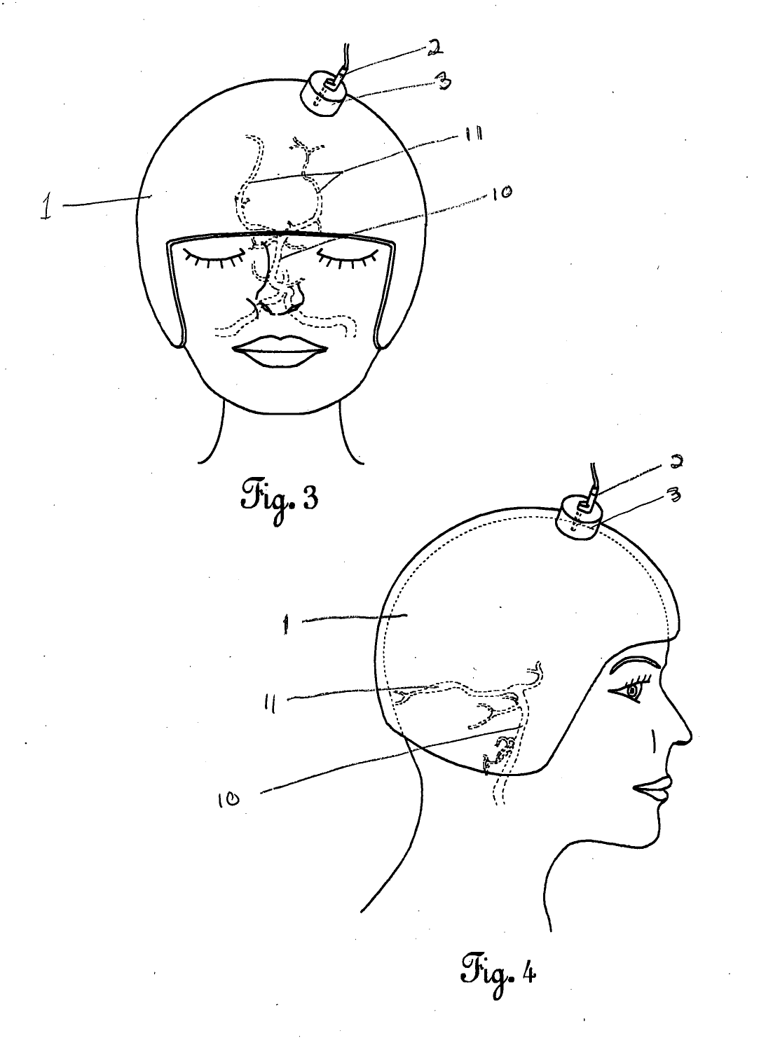

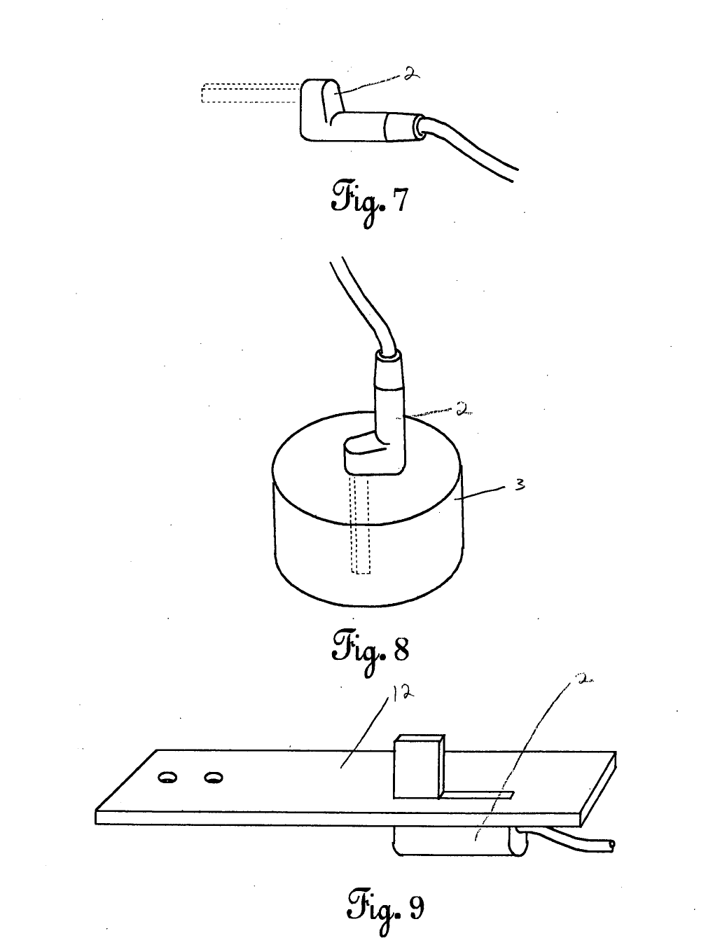

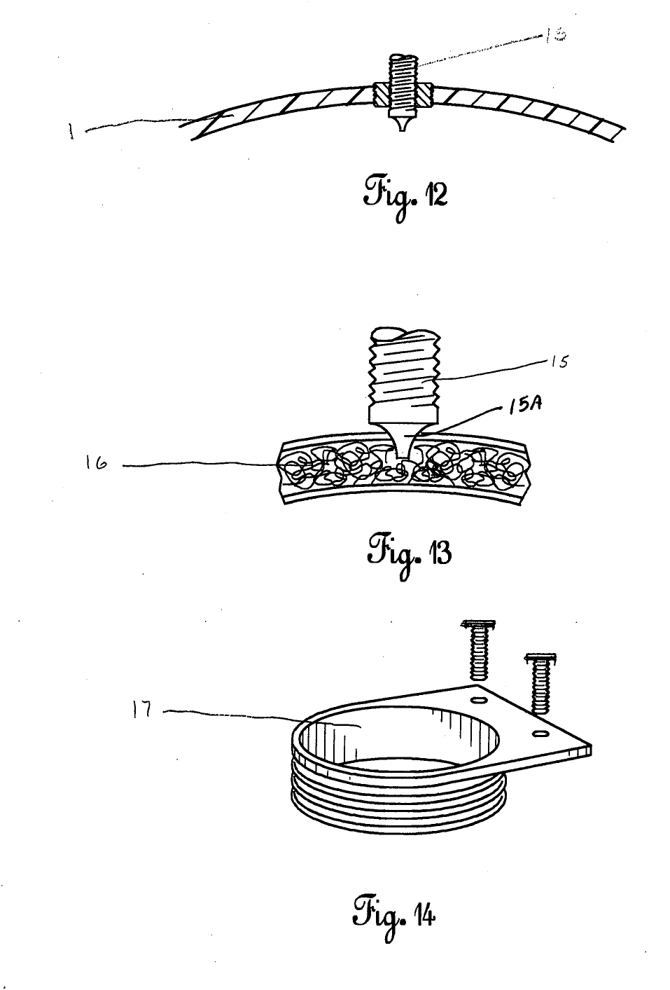

With reference to FIGS. 1-16, it is seen that the helmet (1) is made to fit on the head of one specific person, and that it is secured to that person's head with skull pins (15), and that the precise position of the helmet is dictated by the orientation lines (14) inscribed on the surface of the helmet (1).

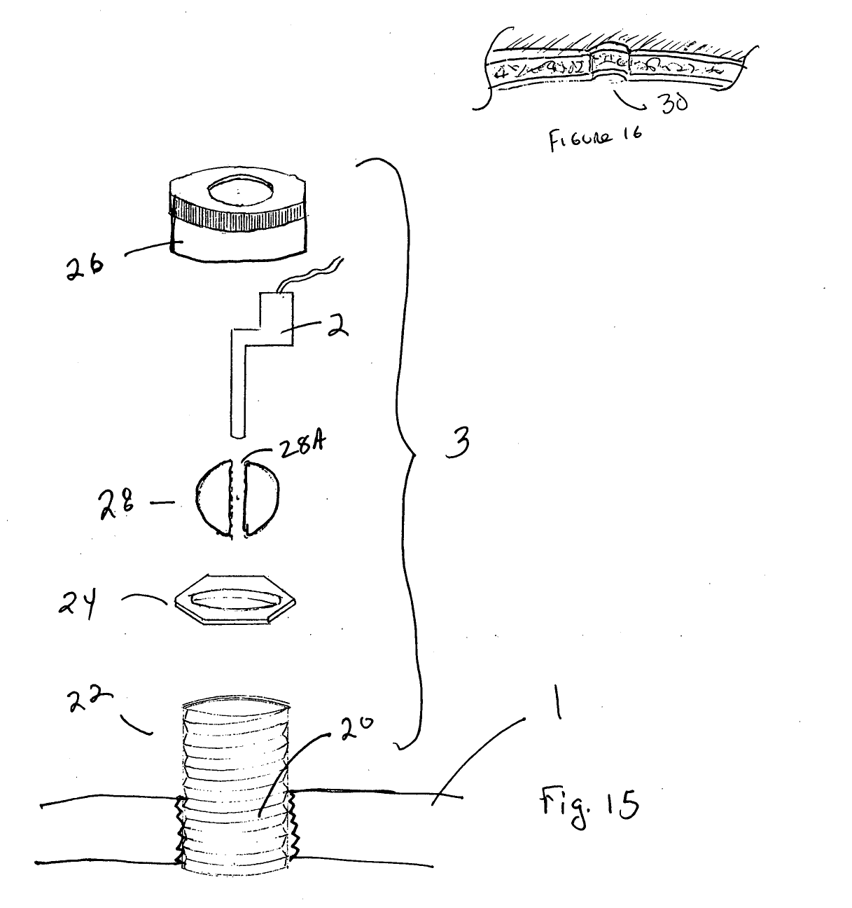

It is further seen that the helmet (1) may typically be fitted with several types of adapters (3) (12) that hold Doppler probes (2) secured to predetermined windows (20) on the helmet (1). These adapters or brackets secure Doppler probes (2) to the helmet (1); and can fix a probe (2) anywhere within a few millimeters of the center of the window at an angle typically within a few degrees of the axis of the window; in order to precisely line up with the flow vector of the artery that the probe (2) is monitoring. Adapters or brackets may thread into the helmet (FIG. 1A) and hold the probe by friction fit or other ways. The adapters or brackets may be constructed as in FIG. 15 to adjustably mount the probes with respect to the helmet.

In current use is the machinery to produce accurate physical models of a patient's skull and intracranial arteries derived from the data obtained from medical imaging studies, i.e. a digital CAT scan or MRI scan. Once the raw data is obtained, specialized software is used to construct a virtual 3D model. This is fed to a prototyping machine that produces a final model of the helmet in physical form. The same technology and machinery can also be used to make a shell (helmet) to fit a patient's head. The virtual model will also identify the exact spatial relationship between the flow vectors of the major intracranial arteries and the surface of the helmet (1). The prototyping machine will make a helmet (1) with precut windows precisely in line with those flow vectors. The lab will typically provide the adaptors to attach the probes to the helmet at the window sites and will tap the holes for fasteners.

Windows are simply round holes cut through the helmet. The site of the window is accurately determined by the computer lab.

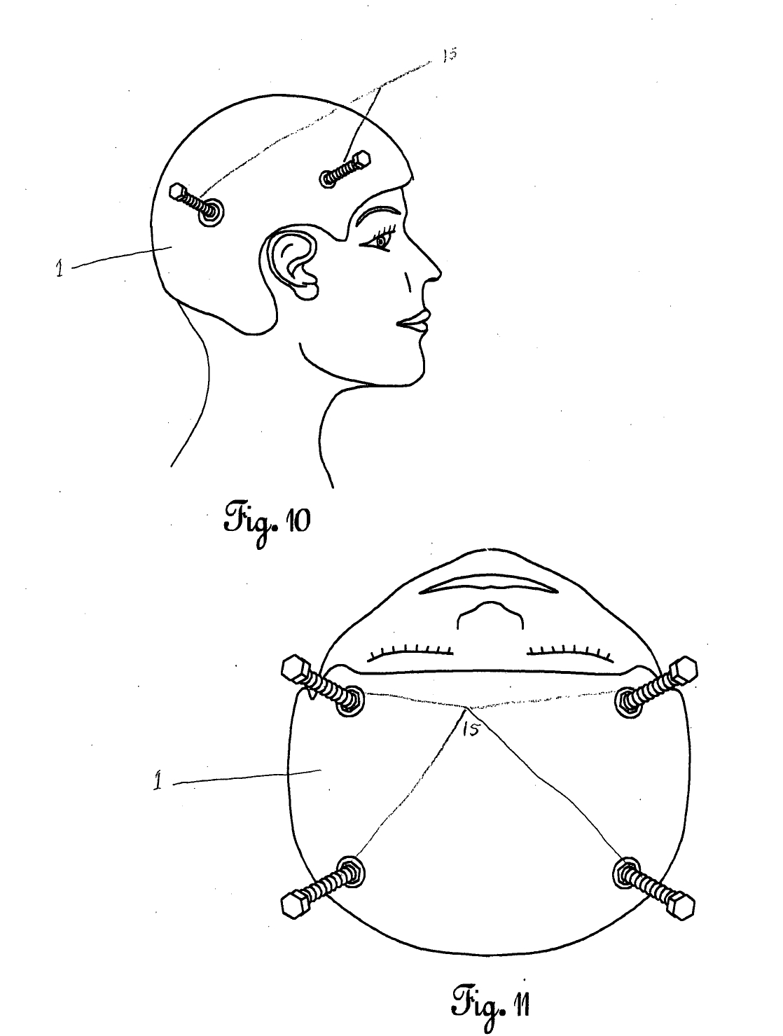

Separate from the windows, the lab can set threads in the helmet for skull pins 15, typically four as seen in FIGS. 10-14. Skull pin 15 may be a threaded fastener and would typically have a button tip 15A attached thereto.

The same data can provide coordinates of several skull landmarks that are readily identifiable on a patient, i.e. external auditory canal, nasion, zygoma, mastoid process. When the virtual model of the helmet (1) is made the software can identify these landmarks with respect to an arbitrary point on the helmet surface. When the physical model is made it can be inscribed with surface lines oriented from the arbitrary point toward the landmarks. This will help ensure that the helmet is properly fitted to the patient.

The helmet (1) is then fitted with special mounting adapters (3), that have a tubular part that is inserted through the pre-cut window, and rests gently against the scalp of the user where it is fixed in place in the helmet (1). Each adapter is designed to secure to a window while holding a probe (2) aligned to a flow vector. The adapter (2) is further designed for fine adjustments of the probe (2), such that the probe (2) can be set anywhere within a few millimeters of the center of the window and angled a few degrees with respect to the axis of the window, and then secured in place for continuous monitoring.

Orientation lines 14 in FIG. 6 are inscribed in the helmet when it is made. These lines are directed at anatomical landmarks in order to mount the helmet onto the individual in the desired orientation. The computer identifies the location of the orientation lines 14 and makes a virtual image of the helmet surrounding the head and picks up an arbitrary point on the surface of the helmet (X,Y,Z). It then tilts and rotates the combined images of the head and helmet until it is an identifiable skull landmark and X,Y,Z in the same plane and then inscribes a line along this plane for the fabricating machine to reproduce when constructing the helmet. The computer prepares a few orientation lines having the same origin, thus positioning the helmet with respect to the head and thus being capable of positioning windows thereupon.

When the helmet is placed on the patient's head, it is held lightly in place with the skull pins adjacent the skull. This allows the surgeon a little leeway to move the helmet until he gets it situated where he wants it, using localizing lines to help with initial orientation of the helmet to the head. The Dopplers are then adjusted to obtain an adequate signal from each of the Dopplers and when they can be monitored properly through all windows, the pins are then finally positioned and secured to the patient's head (see FIGS. 11, 12, and 13).

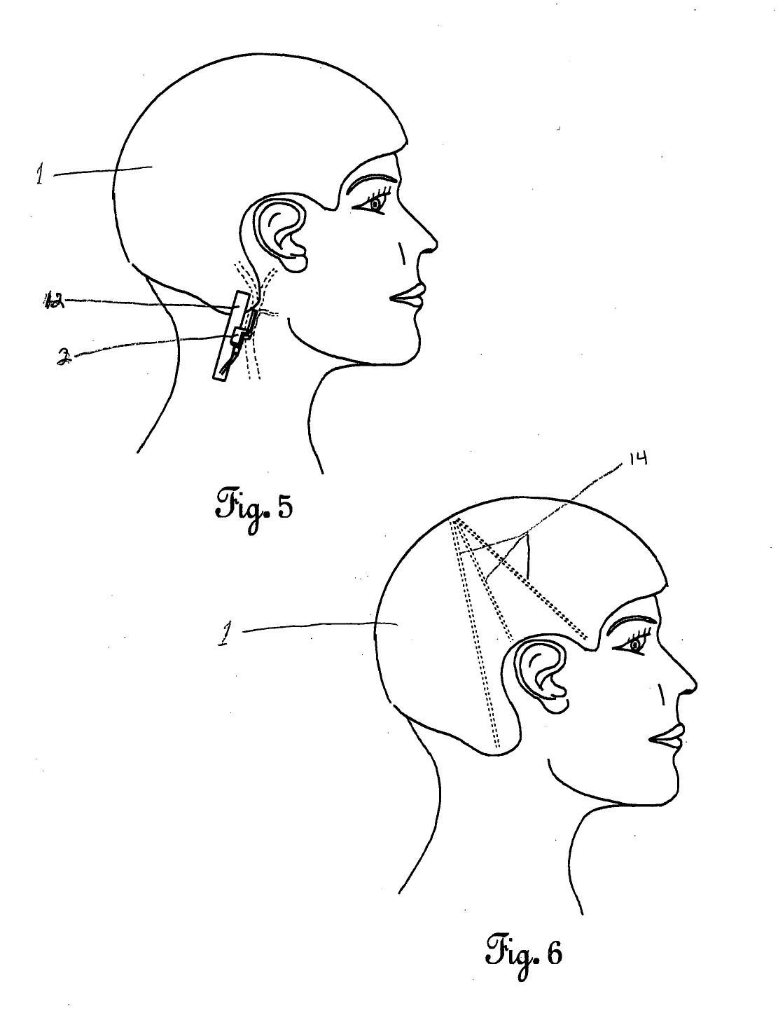

Because the original data typically identifies all the major intracranial arteries, and their flow vectors, the helmet (1) provides the opportunity to monitor several major arteries simultaneously with different probes (2). It is also seen, from FIG. 5 that the left or right extra-cranial portion of the internal carotid artery can also be monitored with a Doppler probe properly mounted and directed.

With further reference to FIG. 15, it is illustrated how adaptors 3 may be adjustably mounted to aim Doppler probes. More specifically, FIG. 15 is seen to illustrate that adaptors 3 could consist of a tube 22 with outside threads that screw into the helmet at each window 20. Since the helmet is typically about ⅛ to ¼ inch thick, the holes could be tapped and threaded when the helmet is made for lab.

When the DMP holder is screwed into the helmet at the desired depth (i.e., with the tube just touching the scalp), a locknut 24 is tightened against the helmet surface securing the tube in place. The probe is then inserted, for example, by a snug friction fit through a channel 28A and a ball 28, which is placed atop the tube and would typically sit atop the tube, but fit within the cap 26. Before tightening the cap, the probe can be rotated and positioned with a few degrees of angulation as needed. The desired position is the one that receives the best signal from the artery being monitored. The arterial segment being mounted is about ½ inch long and the signal will travel 2 to 3 inches.

Typically, the helmet would be initially set up with the DMP monitoring five arteries—both middle cerebral arteries, the basilar artery, the anterior cerebral artery complex, and one extra-cranial internal carotid artery.

The same type of DMP may be used for all sites. The adaptor or adjustable bracket that holds the DMP aligned to the extra-cranial internal carotid artery may be different in that it holds the DMP below the ear and behind the angle of the jaw and points upward, almost tangentially to the skull (see FIG. 5). In an alternate embodiment, the helmet could extend down a bit further and a window may be used.

At the time of setup, the DMPs and the helmet itself will need careful adjustment to assure the DMPs are aligned properly and receiving signals. The helmet is then secured to the skull and the DMPs are locked in place.

FIG. 16 illustrates burr holes 30, such as a burr hole drilled by a ⅝″ drill bit, in the patient's skull, which are provided for and in alignment with the DMPs along flow vectors of the major intra-cranial arteries. These burr holes may be necessitated because current Doppler technology may penetrate an intact skull. These burr holes may be located along the Doppler axis at the time of the initial digital scan and the scalp can be marked appropriate for later cranial ostomies.

Although the invention has been described in connection with the preferred embodiment, it is not intended to be limit to the inventor's particular form set forth, but on the contrary, it is intended to cover such alterations, modifications, and equivalences that may be included in the spirit and scope of the invention as defined by the appended claims.

I claim:

1. A helmet having an inner surface, and an outer surface, the helmet includes multiple holes, or windows, specifically located by reference to data obtained from a computerized tomographic scan of the user's head; with adapters fitted to these windows that secure Doppler probes positioned to face specific major arteries inside and outside of the brain of the user, and aligned to the flow of blood in those arteries directly toward or away form the probes,

2. The helmet according to claim 1 wherein the helmet is also fitted with an adapter that holds a Doppler probe over the extra-cranial portion of an internal carotid artery oriented to monitor the extra-cranial blood flow.

3. The helmet according to claim 1, wherein the position of the windows and adapters as well as the directional settings of the probes has been determined from data obtained from a computerized scan of the users head.

4. The helmet according to claim 1, wherein the helmet is made of a rigid plastic-like substance by computer controlled machinery using the data obtained from a computerized scan of the user's skull and major intracranial arteries.

5. The helmet according to claim 1, wherein the outer surface of the helmet is inscribed with lines that, when extrapolated over the user's head, intersect on specific landmarks of the user's head and skull.

6. The helmet according to claim 1, wherein the several adapters fitted to the helmet are capable of linear and angular adjustments to fix a Doppler probe precisely in line with an arterial flow vector.

7. The helmet according to claim 1, wherein the adapters that are fitted to the helmet have a tubular end that penetrates through the helmet and gently abuts the scalp of the user.
